(19) United States
(12) Patent Application Publication
(10) Pub. No.: US 2024/0377407 A1
(43) Pub. Date: Nov. 14, 2024

(54) OPTICAL PROBE, BIOSENSOR, PAPER-BASED LATERAL FLOW STRIP ASSEMBLY, AND METHOD FOR DETECTING AN ANALYTE

(71) Applicant: UNIVERSITY OF MASSACHUSETTS, Boston, MA (US)

(72) Inventors: Nianqiang Wu, Amherst, MA (US); Yingjie Hang, Amherst, MA (US); Weirui Tan, Amherst, MA (US)

(21) Appl. No.: 18/659,684

(22) Filed: May 9, 2024

Related U.S. Application Data
(60) Provisional application No. 63/465,940, filed on May 12, 2023.

(51) Int. Cl. G01N 33/58; B82Y 15/00; B82Y 30/00; G01N 33/543

(52) U.S. Cl. CPC G01N 33/587; B82Y 15/00; B82Y 30/00; G01N 33/54388

(57) ABSTRACT

The present disclosure is directed to optical probes. An optical probe can include a plasmonic core, a buffer layer on the plasmonic core, and a composite shell comprising a fluorophore embedded in a transparent metal oxide matrix. An optical probe can include a plasmonic core, a transparent shell encapsulating the core, and a plurality of Raman dyes proximate to the core and embedded in the transparent shell. An optical probe can include a plasmonic core comprising a nanoparticle; and a plurality of secondary nanoparticles proximate to the plasmonic core selected to provide plasmonic coupling to the plasmonic core by charge transfer. The optical probes can be useful in various detection systems, for example paper-based lateral flow strip assemblies, particularly where it is necessary to detect low levels of an analyte.

### CROSS REFERENCE TO RELATED APPLICATION

This application claims priority to U.S. Provisional Patent Application No. 63/465,940, filed on May 12, 2023, the contents of which are hereby incorporated by reference herein in their entirety.

### FEDERAL RESEARCH STATEMENT

This invention was made with government support under Grant No. U01 NS119647-01 and Grant No. 3U54EB007958-15S1, both awarded by the National Institute of Health. The government has certain rights in the invention.

### BACKGROUND

Protein biomarkers are important health indicators which can reflect various human health conditions. For example, they are widely used to detect diseases for early intervention and monitoring of the effectiveness of medical treatment. Protein biomarkers are typically measured by enzyme-linked immunosorbent assays (ELISA) and western blots. These techniques require sophisticated instruments and professional personnel in a central laboratory, causing tedious work, long turnover time, and financial burden on patients as well as travel to hospitals. These factors have prompted the development of rapid, inexpensive, and easy-to-operate point-of-care testing (POCT) devices to shift the detection paradigm from central laboratories and hospitals to home and fields of patient care.

Accordingly, there remains a need for improved point-of-care testing, for example of various protein biomarkers.

### SUMMARY

An aspect of the present disclosure is an optical probe comprising: a plasmonic core comprising a nanoparticle, the nanoparticle comprising gold, silver, copper, or a combination thereof; a buffer layer on a surface of the plasmonic core, wherein the buffer layer is transparent and insulating; and a composite shell on the buffer layer, the composite shell comprising a plurality of fluorophores embedded in a transparent metal oxide matrix.

Another aspect is an optical probe comprising: a plasmonic core comprising a nanoparticle, the nanoparticle comprising gold, silver, copper, or a composition thereof; a transparent shell on the plasmonic core, the transparent shell comprising a transparent ternary metal oxide; and a plurality of Raman dyes proximate to the plasmonic core and embedded in the transparent shell.

Another aspect is an optical probe comprising: a plasmonic core comprising a nanoparticle, the nanoparticle comprising gold or silver; and a plurality of secondary nanoparticles proximate to the plasmonic core to provide plasmonic coupling by charge transfer, provided that when the plasmonic core comprises gold, the secondary nanoparticles comprise silver; and when the plasmonic core comprises silver, the secondary nanoparticles comprise gold.

An article comprising the optical probe represents another aspect of the present disclosure.

Another aspect is a method of detecting a target species, the method comprising: introducing an analyte mixture to a detection system comprising the optical probe or the article comprising the optical probe.

Another aspect is a plasmonic nanoarray-containing biosensor, the plasmonic nanoarray-containing biosensor comprising: the optical probe, wherein the optical probe is functionalized with a plurality of detection moieties; a plasmonic nano-array pattern or a plasmon gap mode pattern, each functionalized with a plurality of capture antibodies; wherein the optical probe is capable of binding to the plasmonic nano-array pattern or the plasmon gap mode pattern upon contact with an analyte.

Another aspect is a method of detecting a target species, the method comprising: introducing an analyte mixture comprising the target species to a detection system comprising the plasmonic nanoarray-containing biosensor.

Another aspect is a paper-based lateral flow strip assembly comprising the optical probe, the plasmonic nanoarray-containing biosensor, or both.

Another aspect is a method of detecting an analyte, the method comprising: contacting a human fluid sample, preferably a whole blood sample, a blood plasma sample, a saliva sample, a nasal swab sample, or a urine sample, with the paper-based lateral flow strip assembly.

The above described and other features are exemplified by the following figures and detailed description.

### BRIEF DESCRIPTION OF THE DRAWINGS

The following figures represent exemplary embodiments.

FIG. 1 is a schematic illustration of an optical probe according to an aspect of the present disclosure.

FIG. 2 is a schematic illustration of an optical probe according to an aspect of the present disclosure.

FIG. 3 is a schematic illustration of an optical probe according to an aspect of the present disclosure.

FIG. 4 is a schematic illustration of an optical probe according to an aspect of the present disclosure.

FIG. 5 is a cross-sectional view of an illustration of a chip-based optical detection platform according to an aspect of the present disclosure.

FIG. 6 is an illustration of a chip-based optical detection platform according to an aspect of the present disclosure.

FIG. 7 is an illustration of a nano-array chip-based detection platform according to an aspect of the present disclosure.

FIG. 8 is an illustration of a nano-array chip-based detection platform according to an aspect of the present disclosure.

FIG. 9 is an illustration of an optical detection platform according to an aspect of the present disclosure.

FIG. 10 is an illustration of a paper-based lateral flow strip (PLFS) for detection of an analyte.

FIG. 11 is an illustration of a double-layer nanofiber membrane for blood plasma separation.

FIG. 12 is an illustration of a paper-based lateral flow strip (PLFS) including a heater.

FIG. 13 is an illustration of a paper-based lateral flow strip assembly for multiplexed detection of multiple analytes in single strip.

FIG. 14 is an illustration of a paper-based lateral flow strip assembly for multiplexed detection of multiple analytes in two strips.

FIG. 15 is an illustration of a paper-based lateral flow strip assembly for multiplexed detection of multiple analytes in two strips.

FIG. 16 is an illustration of a paper-based lateral flow strip (PLFS) for detection of an analyte using chemiluminescence.

FIG. 17 is a plot of NSE concentration (ng/ml) versus Raman intensity (a.u.).

FIG. 18 is a plot of p24 antigen (Log([p24](pg/mL) versus Raman intensity (a.u.).

### DETAILED DESCRIPTION

Point-of-care testing tools such as colorimetric paper lateral flow strips (PLFS) are anticipated to boost the growth of the market due to their low cost, fast response, portability, and case of use. Nevertheless, colorimetric PLFS suffer from poor sensitivity and severe interference from complex human fluid sample matrices, especially red blood cells in whole blood samples. For example, paper substrates are vulnerable to visible interference with the strong red color from red blood cells, which can cause errors in test results. In particular, erythrocytes, icterus, and hemolysis with absorption in the optical detection zone can substantially reduce the sensing signal quality. To remove these molecules away from the detection area, extra washing steps are required, which increases the sample-to-answer time. Therefore, for whole blood testing, it is necessary to remove red blood cells from the whole blood sample matrix before the fluid reaches the detection zone in the PLFS. This is why sensing transduction and detection are generally performed in blood plasma rather than whole blood. Conventional strategies for separation of plasma from whole blood, such as mechanical forces, electro-osmotic flow, dielectrophoretic techniques, and magnetic interactions, can only be carried out in central laboratories or patient-care centers where sophisticated instruments and specialized laboratory personnel are available. These methods cannot meet the need of whole blood testing in point-of-care settings. Thus, significant efforts have been devoted to the development of “on-chip” or “on-strip” plasma separation assembles to enable direct whole blood testing with miniaturized point-of-care testing tools.

To address the above-described technical limitations, the present inventor has discovered near-infrared (NIR) fluorescence or surface-enhanced Raman scattering (SERS) can be used for sensing signal transduction in a PLFS to improve sensitivity and to enable quantification of analytes. NIR-fluorescence or NIR-SERS can “penetrate” blood (or other human fluids) and has a strong resistance to interference from sample matrices, which greatly enhances direct detection of analytes. To enhance the fluorescence or SERS intensity, a plasmonic nanostructured NIR-fluorescence probe or SERS probe has been developed. In a further advantageous feature, a nanostructured chip can be included in the test line/zone to further enhance the intensity of NIR-fluorescence or SERS. These combined nanotechnologies result in high sensitivity and low limit of detection (LOD). To realize automation and miniaturization, the fluorescence or SERS probes can be incorporated into a paper-based microfluidic strip to form an integrated PLFS. A blood plasma separation unit can be integrated into the PLFS to form a miniaturized POCT device, which shortens the time for sample processing, allowing for rapid detection of biomarkers (e.g., HIV, SARS-Cov-2, cancer, Alzheimer's disease, and traumatic brain injury (TBI) biomarkers) with a small volume of sample (e.g., a whole blood sample), eliminating the need for sample pretreatment in a central laboratory prior to detection. If the PLFS is used for saliva or nasal swab samples, the plasma separation unit can be replaced with a membrane filter. Thus, the integrated PLFS according to the present disclosure is capable of sensitively and quantitatively measuring proteins in saliva, nasal swab samples, urine, and finger-prick blood samples at home, in clinics, and in resource-limited settings. A significant improvement is therefore provided by the present disclosure.

Accordingly, an aspect of the disclosure is an optical probe.

In an aspect, an optical comprises a plasmonic core, a buffer layer on a surface of the plasmonic core, and a composite shell on the buffer layer.

The plasmonic core comprises gold, silver, copper, or a combination thereof. In an aspect, the plasmonic core comprises gold. In an aspect, the plasmonic core comprises copper and silver. The plasmonic core can have any suitable shape for example, nanospheres, nanocubes, nanorods, or nanostars. In an aspect, the plasmonic core can comprise porous nanoparticles. In an aspect, the plasmonic core can be bimetallic. As used herein, the term “bimetallic plasmonic core” refers to a core having two different metals present. For example, a bimetallic plasmonic core can comprise gold nanostars decorated with silver nanodots on the surface, bimetallic gold-silver nanoparticles (e.g., nanocubes), or porous bimetallic gold-silver nanoparticles (e.g., nanocubes). A combination of bimetals will allow for the plasmonic core to exhibit a localized surface plasmon resonance (LSPR) band in either near-infrared I (NIR I) window (700-900 nm) or NIR II window (1000-1300 nm). This will enable a paper-based lateral flow strip comprising the bimetallic plasmonic core to be used as a SERS-based test strip under excitation of a 785 nm laser or a 1064 nm laser.

The size (e.g., diameter) of the plasmonic core can be, for example, 10 to 200 nanometers, or 15 to 180 nanometers, or 15 to 100 nanometers, or 15 to 70 nanometers. The term “diameter” is used herein to refer to the size of the plasmonic core, however, it will be understood that when the plasmonic core has a non-spherical shape, the term “diameter” refers to the longest cross-sectional dimension of the core.

In an aspect, the plasmonic core can have an extinction peak in a spectral range of 700 to 850 nanometers. In an aspect, the plasmonic core can have an extinction peak in a spectral range of 1,000 to 12,000 nanometers. In an aspect, the plasmonic core can have an extinction peak in a spectral range of 400 to 670 nanometers. In an aspect, the extinction peak of the plasmonic core at least partially overlaps with the absorption peak or emission peak of the fluorophore of the composite shell. In an aspect, the wavelength of the excitation light at least partially overlaps with the extinction peak of the plasmonic core.

A buffer layer encapsulates the plasmonic core. The buffer layer is preferably a transparent layer. The term “transparent” as used herein means that the buffer layer has a substantial absence of cloudiness such that light can pass through in a substantially unobstructed manner. The buffer layer is further preferably an insulating layer. Exemplary buffer layers can comprise, but are not limited to, metal oxides such as silica, sodium- or potassium-doped silica, alumina, zinc oxide, tin oxide, indium oxide, and the like or a combination thereof. In an aspect, the buffer layer comprises silica or alumina.

The buffer layer can completely surround the plasmonic core and can have a thickness of, for example, 1 to 40 nanometers, or 2 to 20 nanometers.

A composite shell is disposed on and encapsulates the buffer layer. The composite shell comprises a plurality of fluorophores embedded in a transparent metal oxide matrix. Exemplary fluorophores can include organic dyes, inorganic semiconducting quantum dots, chalcogenides, graphene oxide quantum dots, carbon dots, metal-organic framework (MOF), or a combination thereof. In an aspect, the fluorophore comprises an organic dye, for example a cyanine dye. In an aspect, the fluorophore comprises a plurality of quantum dots, for example comprising cadmium, tellurium, selenium, or a combination thereof. In an aspect, the quantum dots can comprise CdSe quantum dots, CdTe quantum dots, or CdTeSe quantum dots.

In an aspect, the fluorophore can have an emission peak of 700 to 850 nanometers. In an aspect, the fluorophore can have a peak of 1000 to 1200 nanometers. In an aspect, the fluorophore can have an emission peak of 400 to 670 nanometers.

The composite shell can completely surround the buffer layer and can have a thickness of, for example, 1 to 25 nanometers, or 2 to 20 nanometers.

The metal oxide of the buffer layer and the matrix of the composite shell can be the same or different. In an aspect, the metal oxide of the buffer layer and the matrix of the composite shell are the same. In an aspect, the metal oxide of the buffer layer and the matrix of the composite shell can be either silica or alumina. In an aspect, one or both of the buffer layer and the composite shell can comprise a ternary metal oxide. The ternary metal oxide can be of the formula MₓSi₁₋ₓO₂, or M₂ₓAl₂₋ₓO₃, wherein x is 0 to 0.5, preferably 0.01 to 0.5 and M is Mg, Ba, Sr, Ca, K, Na, or a combination thereof. Without wishing to be bound by theory, the ternary metal oxides can offer more tunability compared to binary metal oxides for thickness and/or conformability.

The optical probe can have an overall size of 12 to 250 nanometers, or 15 to 200 nanometers, or 15 to 150 nanometers, or 19 to 110 nanometers.

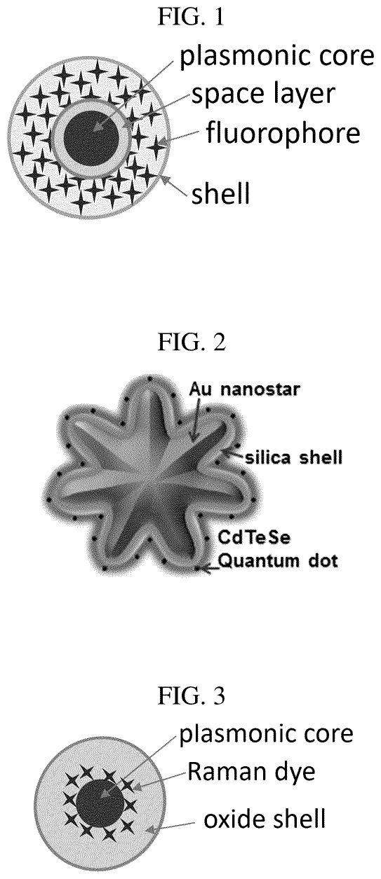

In a specific aspect, the optical probe can comprise a plasmonic core comprising gold nanostars, a buffer layer comprising silica, and a composite shell comprising silica and an organic fluorescent dye. The organic fluorescent dye can comprise a cyanine dye. The core can have a diameter of 10 to 70 nanometers. The buffer layer can have a thickness of 2 to 20 nanometers. The composite shell can have a thickness of 2 to 20 nanometers. An exemplary optical probe is shown in the schematic illustration of FIG. 1.

In a specific aspect, the optical probe can comprise a plasmonic core comprising gold nanostars, a buffer layer comprising silica, and a composite shell comprising silica and a plurality of quantum dots. The quantum dots can be CdTeSe quantum dots. The core can have a diameter of 10 to 70 nanometers. The buffer layer can have a thickness of 2 to 40 nanometers. The composite shell can have a thickness of 2 to 20 nanometers. An exemplary optical probe is shown in the schematic illustration of FIG. 2.

Another aspect of the present disclosure is an optical probe comprising a plasmonic core comprising a nanoparticle, a transparent shell on the plasmonic core, and a plurality of Raman dyes proximate to the plasmonic core and embedded in the transparent shell.

The plasmonic core comprises nanoparticles comprising gold, silver, copper, or a composition thereof. In an aspect, the plasmonic core can be bimetallic, comprising at least two of gold, silver, and copper. In an aspect, the bimetallic plasmonic core comprises gold and silver. The bimetallic plasmonic core can have any suitable shape for example, nanospheres, nanocubes, nanorods, or nanostars. In an aspect, the plasmonic core can be bimetallic. In an aspect, the bimetallic plasmonic core can comprise gold nanostars decorated on the surface with silver nanodots, or porous bimetallic gold-silver nanocubes.

The plasmonic core can have an extinction peak of 420 to 850 nanometers.

The size (e.g., diameter) of the plasmonic core can be, for example, 10 to 200 nanometers, or 10 to 180 nanometers, or 15 to 100 nanometers, or 15 to 70 nanometers. The term “diameter” is used herein to refer to the size of the plasmonic core, however, it will be understood that when the plasmonic core has a non-spherical shape, the term “diameter” refers to the longest cross-sectional dimension of the plasmonic core.

A transparent shell is disposed on a surface of the plasmonic core. The transparent shell comprises a transparent ternary metal oxide. The ternary metal oxide can be of the formula MₓSi₁₋ₓO₂, or M₂ₓAl₂₋ₓO₃, wherein x is 0 to 0.5, preferably 0.01 to 0.5 and M is Mg, Ba, Sr, Ca, K, Na, or a combination thereof. Without wishing to be bound by theory, the ternary metal oxides can offer more tunability compared to binary metal oxides for thickness and/or conformability.

The transparent shell can completely surround the bimetallic plasmonic core and can have a thickness of, for example, 1 to 40 nanometers, or 2 to 20 nanometers.

A plurality of Raman dyes are embedded in the transparent shell and located proximate to the bimetallic plasmonic core. Exemplary Raman dyes can include, but are not necessarily limited to, malachite green (MG), mercaptobenzoic acid (MBA), Rhodaminc 6G, 4-[2-[2-Chloro-3-[(2,6-diphenyl-4H-thiopyran-4-ylidene)ethylidene]-1-cyclohexen-1-yl]ethenyl]-2,6-diphenylthiopyrylium tetrafluoroborate (IR1061), or a combination of thereof.

The optical probe can have an overall size of 11 to 225 nanometers, or 15 to 200 nanometers, or 17 to 200 nanometers.

In a specific aspect, the optical probe can comprise a bimetallic plasmonic core comprising gold nanostars decorated with silver nanoparticles on the gold surface, and a transparent shell comprising a transparent ternary metal oxide and a Raman dye between the bimetallic plasmonic core and the transparent shell. The bimetallic plasmonic core can have a diameter of 10 to 180 nanometers. The transparent shell can have a thickness of 2 to 20 nanometers. An exemplary optical probe is shown in the schematic illustration of FIG. 3.

In a specific aspect, the core-shell optical probe can be dual-functionalized with SERS and fluorescence emission if the mixed fluorescence reporter and SERS reporter are contained in the probes or if the Raman dye (e.g., Rhodamine 6G) can serve as both fluorescence reporter and SERS reporter.

Another aspect is an optical probe comprising a plasmonic core comprising a nanoparticle and a plurality of secondary metal nanoparticles disposed proximate to the plasmonic core.

The plasmonic core comprises gold or silver. In an aspect, the plasmonic core comprises gold. In an aspect, the plasmonic core comprises copper and silver. The plasmonic core can have any suitable shape for example, nanospheres, nanocubes, nanorods, or nanostars. In an aspect, the plasmonic core can comprise porous nanoparticles.

The size (e.g., diameter) of the plasmonic core can be, for example, 10 to 200 nanometers, or 15 to 180 nanometers, or 15 to 100 nanometers, or 15 to 70 nanometers. The term “diameter” is used herein to refer to the size of the plasmonic core, however, it will be understood that when the plasmonic core has a non-spherical shape, the term “diameter” refers to the longest cross-sectional dimension of the core.

In an aspect, the plasmonic core can have an extinction peak in a spectral range of 700 to 850 nanometers. In an aspect, the plasmonic core can have an extinction peak in a spectral range of 1,000 to 12,000 nanometers. In an aspect, the plasmonic core can have an extinction peak in a spectral range of 400 to 670 nanometers. In an aspect, the extinction peak of the plasmonic core at least partially overlaps with the absorption peak or emission peak of the fluorophore of the composite shell. In an aspect, the wavelength of the excitation light at least partially overlaps with the extinction peak of the plasmonic core.

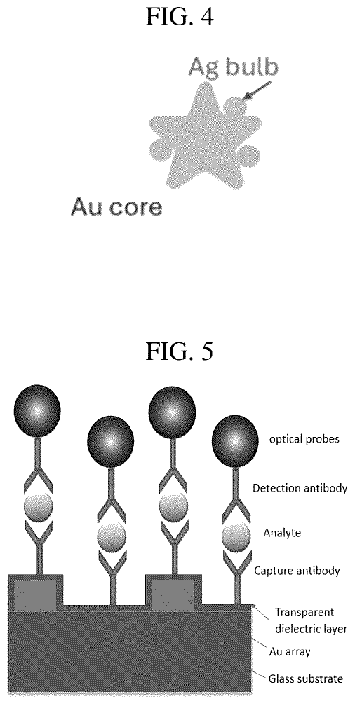

The surface of the plasmonic core is decorated with a plurality of metal nanoparticles selected to provide SERS signals. The secondary nanoparticles are selected to provide plasmonic coupling to the plasmonic core by charge transfer. Accordingly, when the plasmonic core comprises gold, the secondary nanoparticles comprise silver, and when the plasmonic core comprises silver, the secondary nanoparticles comprise gold. In an aspect, the nanoparticles are silver nanoparticles disposed proximate to the plasmonic core. In an aspect, the nanoparticles are gold nanoparticles disposed proximate to the plasmonic core. In an aspect, adjacent nanoparticles are separated by a gap of at least 10 nanometers. The decorating nanoparticles can preferably be spherical in shape, and can have, for example, an average diameter of 3 to 30 nanometers. In an aspect, the optical probe can optionally further comprise an encapsulating shell coated on the surface. Without wishing to be bound by theory, it is believed that presence of such a shell can improve the stability of the probe and can provide opportunities for further functionalization of the probe, if desired (e.g., with an antibody, an aptamer, or functional group such as carboxylic acid). In an aspect, when present, the encapsulating shell can comprise silica. An exemplary optical probe is shown in the schematic illustration of FIG. 4.

Articles comprising the optical probes disclosed herein represent another aspect of the present disclosure. For example, the above-described optical probes can be particularly useful in various detection platforms, particularly wherein the sample to be analyzed is a bio-fluid such as blood, plasma, urine, saliva, or buffers.

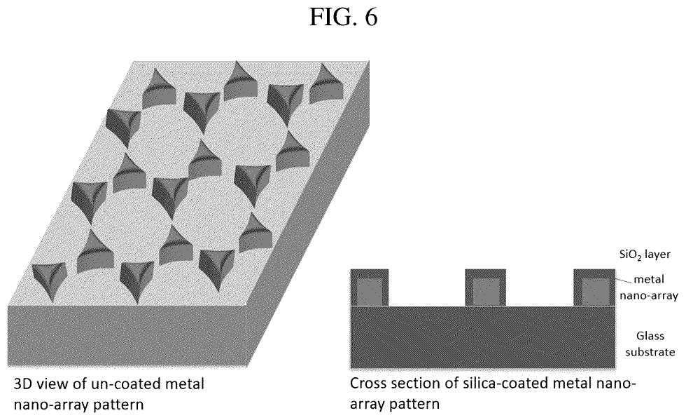

The optical probes can be useful in a nano-array chip-based detection platform. Such a chip-based optical detection platform can be as shown in FIG. 5 or FIG. 6, wherein an assembly comprises a nanopatterned chip, a first molecular recognition element (e.g., also referred to herein as a “detection moiety” such as a capture antibody or a capture aptamer), an analyte (e.g., a protein, a small molecule, etc.), a second molecular recognition element (e.g., a detection antibody or a detection aptamer), and one or more of the optical probes described herein.

In a specific aspect, the nano-array chip-based optical detection platform can comprise a substrate, for example, a substrate (e.g., glass, silicon, or the like), with a nanopatterned array (also referred to herein as a “nano-array”) disposed thereon. The nanopatterned array can comprise gold, silver, copper, or a combination thereof. Advantageously, the nanopatterned array can exhibit long range ordering, for example, over a surface area of greater than or equal to 5 mm². A transparent dielectric coating is disposed on the nanoarray-decorated substrate, preferably comprising silica or alumina. The transparent dielectric layer can have a thickness of, for example, 0.3 to 40 nanometers, and can be deposited by any suitable deposition technique, for example, atomic layer deposition. Capture antibodies decorate the transparent dielectric layer, which are capable of interacting with the analyte of interest and can be selected according to the particular analyte. The optical probes can be functionalized with the detection antibody, which can also interact with the captured analyte.

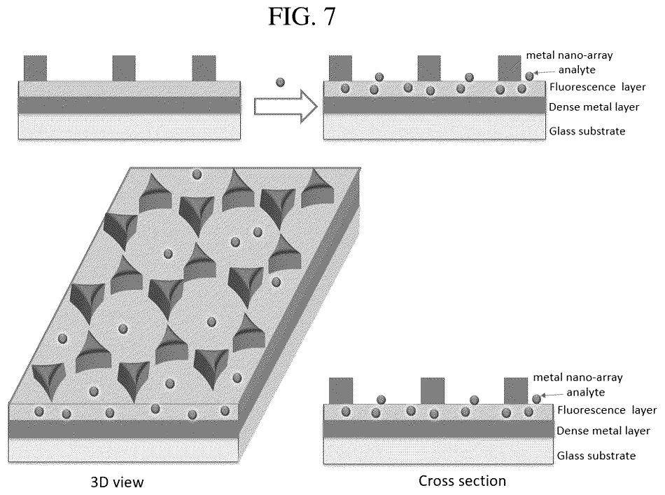

In an aspect, the nano-array chip-based detection platform according to the present disclosure can be a multilayer structure comprising a metal nano-array on a fluorescence layer, and a metal layer on the fluorescence layer on a side opposite the metal nano-array. The metal layer can comprise, for example, gold, silver, or copper, and can be disposed on a supporting layer, for example a glass or silicon substrate. The metal nano-array can comprise a gold or silver metal nano-array. The nano-array chip-based detection platform according to this aspect can be used for detection of various analytes, for example small molecules or metal ions. The fluorescence layer is a semiconductor, for example, a metal-organic framework (MOF) (e.g., a porphyrin MOF). Advantageously, the nano-array chip-based detection platform according to the present aspect provides the fluorescent semiconductor as a dielectric layer between the upper nano-array pattern and the metal layer, and further, coherent plasmon-exciton coupling can be achieved in this structure, leading to significant enhancement of fluorescence emission from the fluorescence layer. It can be challenging to control the thickness and dielectric constant of the fluorescence layer to achieve strong coupling of plasmon-exciton. The thickness of the fluorescence layer can be controlled via atomic layer deposition (ALD) or layer-by-layer self-assembly. An exemplary detection assembly according to this aspect of the disclosure is shown in FIG. 7.

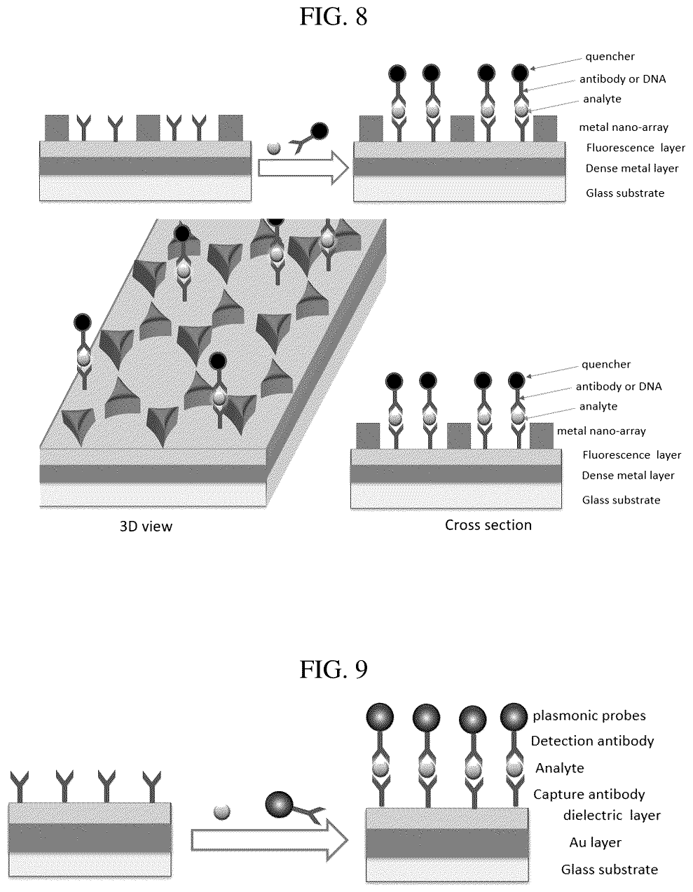

In an aspect, the nano-array chip-based detection platform according to the present disclosure can be based on a fluorescence resonance energy transfer (FRET) mode coupled to a plasmonic gap mode. The plasmonic gap mode assembly comprises of a metal layer (e.g., gold, silver, or copper) on a supporting layer (e.g., a glass or silicon substrate), a fluorescence layer on the metal layer on a side opposite the supporting later, and a metal nano-array pattern (e.g., comprising gold or silver) disposed on the fluorescence layer on a side opposite the metal layer. The fluorescence layer is a semiconductor, for example, a metal-organic framework (MOF) (e.g., porphyrin MOF), a metal chalcogenide (e.g., WS₂, WSe₂, MoS₂, MoSe₂, Cu₂S, CuS, Bi₂S₃, or Bi₂₋ₓCuₓS₃), semiconducting quantum dots embedded in a silica or alumina layer, or fluorescence dye molecules embedded in a silica or alumina layer. When analytes are present in the assay, quenchers (e.g., gold nanoparticles or fluorescence dye molecules) are coupled to the plasmonic gap mode, enabling the FRET process, and leading to quenching of fluorescence emission of the fluorescence layer. Advantageously, the nano-array chip-based detection platform according to the present aspect provides the fluorescent semiconductor as a dielectric layer between the upper nano-array pattern and the metal layer, and further, coherent plasmon-exciton coupling can be achieved in this structure, leading to a significant enhancement of fluorescence emission from the fluorescence layer. It can be challenging to control the thickness and dielectric constant of the fluorescence layer to achieve strong coupling of plasmon-exciton. The thickness of the fluorescence layer can be controlled via atomic layer deposition (ALD) or layer-by-layer self-assembly. The enhanced fluorescence of fluorescent layer excited by the plasmon gap mode is quenched via the FRET process. An exemplary detection assembly according to this aspect of the disclosure is shown in FIG. 8. As shown in FIG. 8, a fluorescent layer is between the metal nano-array and the metal layer.

In an aspect of a plasmon gap mode detection platform, a detection assembly can comprise a metal layer comprising gold, silver, or copper on the glass substrate, with the dielectric layer disposed thereon. The metal layer takes the place of the above-described nanopatterned array. Detection can similarly be accomplished by interaction of the optical probe with the analyte, and coupling of the optical probe-analyte moiety with the substrate through the presence of a capture antibody. Without wishing to be bound by theory, the detection assembly of this aspect may provide improved detection sensitivity. An exemplary detection assembly according to this aspect of the disclosure is shown in FIG. 9. As shown in FIG. 9, plasmon-enhanced fluorescence probes can be coupled to the dielectric oxide-Au double-layer via a detection antibody/analyte/capture antibody ensemble. The dielectric oxide can be silica or alumina. This structure will generate a plasmon gap mode, which improves the detection sensitivity. Unique to the design of the present disclosure is that strong coupling can be achieved between the exciton of the fluorescence probes and the plasmonic gap mode of the dielectric layer, leading to remarkable enhancement of fluorescence emission from the fluorescence probes. It can be challenging to control the thickness of the dielectric oxide layer to achieve strong coupling of plasmon-exciton. The thickness of the dielectric oxide layer can be controlled via atomic layer deposition (ALD).

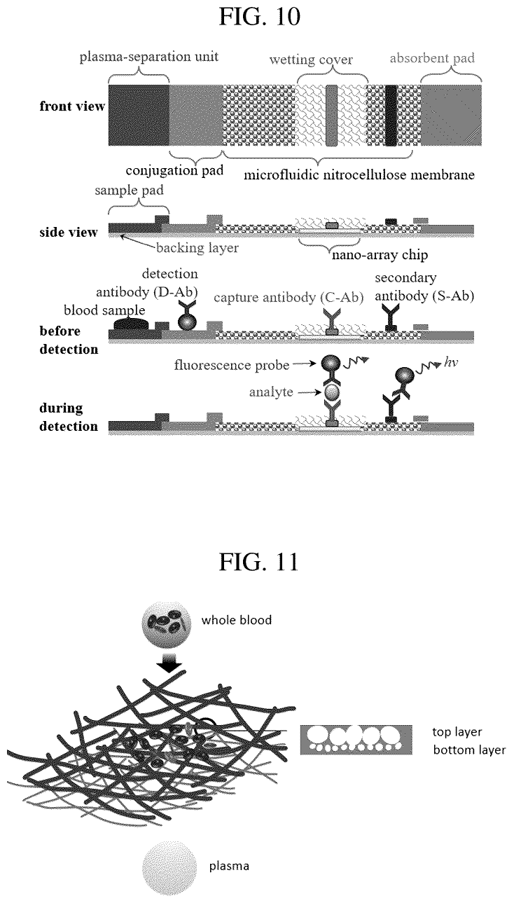

Another aspect of the present disclosure is a paper-based lateral flow strip assembly. The paper-based lateral flow strip assembly comprises the optical probe, the plasmonic nanoarray-containing biosensor, or both. An exemplary paper-based lateral flow strip assembly is shown in FIG. 10.

As shown in FIG. 10, a paper-based lateral flow strip assembly can comprise a plasma separation unit; a conjugation pad comprising a plurality of optical probes, wherein each optical probe is labelled with a detection moiety (also referred to as a “molecular recognition element”), preferably a detection antibody or aptamer; a detection unit (or test line) comprising a capture antibody or aptamer capable of interacting with an analyte that has reacted with the optical probe, and a control line comprising a secondary antibody; and an absorbent pad adjacent to the membrane. The assembly can comprise a suitable paper membrane, for example a nitrocellulose paper membrane. The paper membrane can be on a backing layer, preferably a plastic backing layer. In some aspects, the optical probes can be replaced with quantum dots (e.g., CdSe/ZnS, Ag₂S and other semiconducting quantum dots with visible-light and near-infrared fluorescence emission) or an organic fluorescent dye.

As shown in FIG. 10, a paper-based lateral flow strip assembly can comprise a plasma separation unit; a conjugation pad comprising the optical probe, wherein the optical probe is labelled with a detection moiety, preferably a detection antibody or aptamer; a detection unit comprising nano-array chip functionalized with capture antibodies or aptamers. The capture antibodies or aptamers are capable of interacting with an analyte that has reacted with the optical probe, and a second section comprising a secondary antibody or aptamer; and an absorbent pad adjacent to the detection unit. The assembly can comprise a suitable membrane, preferably a microfluidic membrane, more preferably comprising paper, for example a nitrocellulose paper membrane. The membrane can be on a backing layer, preferably a plastic backing layer. It is noted that in the paper-based lateral flow strip, the nano-array chip in the detection unit can be according to any of the aspects described herein, for example as shown in any of FIG. 5, 7, 8, or 9.

In an aspect of a paper-based lateral flow strip comprising a nano-array chip on the detection zone, a top surface of the chip can be coated by a wetting cover to allow for a liquid to flow over the chip surface. In an aspect, the wetting cover can be dissolved in the liquid. The wetting cover can comprise a mixture of a surfactant (e.g., sorbitan monolaurate, available as Span-20, polysorbate 20, available as Tween-20, and the like, or a combination thereof) and a sugar (e.g., glucose, fructose, sucrose, and the like, or a combination thereof).

In an aspect, in a method of using the paper-based lateral flow strip assembly, a whole blood sample can be placed on the sample pad (also referred to as the plasma separation unit). In an aspect, the plasma separation unit can comprise a functionalized porous nanofiber membrane, for example as shown in FIG. 11. Suitable porous nanofiber membrane can be prepared by electrospinning, oxygen plasma treatment of the electrospun web, and surface functionalization. Suitable polymer materials for preparing the membranes by electrospinning can include, but are not limited to, polyvinylpyrrolidone (PVP), poly(ethylene glycol) (PEG) poly(ethylene oxide) (PEO), poly(lactide) (PLA), polylactic-co-glycolic acid (PLGA), poly(vinyl alcohol) (PVA) and poly(ε-caprolactone) (PCL), polysulfone (PSF), sulfonated-PSA, or a combination thereof. The diameter of nanofibers can be, for example, 50 nm to 300 nm. Double-layer or single-layer membrane are both contemplated by the present disclosure. For double-layer membranes, the pore size of the top-layer (also referred to as the “upstream layer”) can be 2 to120 micrometer, and the pore size of the bottom-layer (also referred to as the “downstream layer”) can be 1 to 5 micrometers. For single-layer membranes, the pore size can be 1 to 120 micrometers.

The electrospun nanofiber membranes can be treated with oxygen plasma to enhance hydrophilicity of membranes. Oxygen plasma oxidizes the surface of polymer nanofiber, generating functional groups such as —OH and —COOH.

The nanofiber membranes can be immersed into an aqueous solution of 0.05 to 2 M salts. The salts can be KCl, NaCl, CaCl₂, KNO₃, NaNO₃, Ca(NO₃)₂, KSO₄, NaSO₄, K₃PO₄, Na₃PO₄, K₂HPO₄, Na₂HPO₄, or a combination thereof. The membrane is then dried to provide the plasma separation unit.

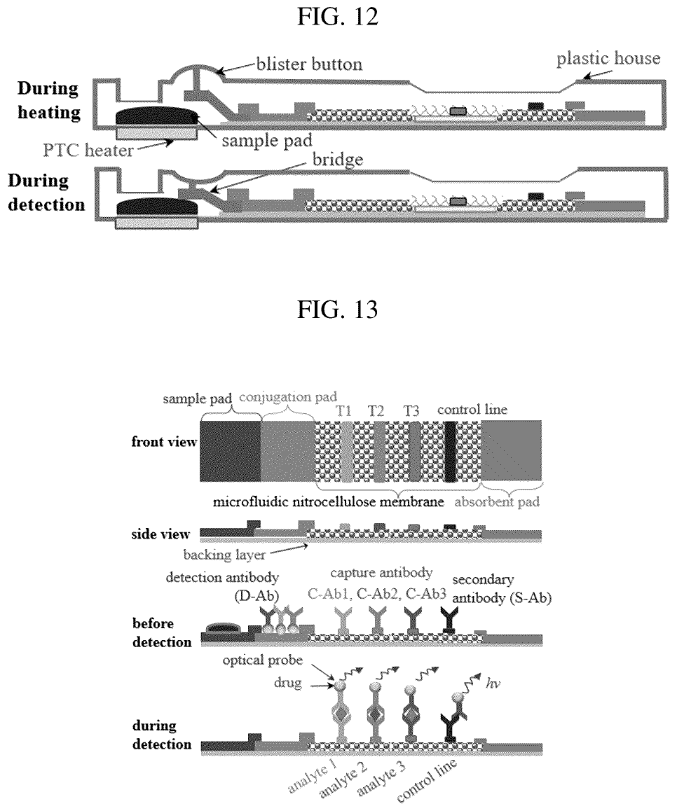

In an aspect, the paper-based lateral flow strip assembly can further comprise a heater, preferably positioned prior to the plasma separation unit, as shown in FIG. 12. As shown in FIG. 12, a heater can be added to the assembly on the backside of the sample pad. During the heating, a bridge capable of connecting the heated sample pad to the rest of the assembly is detached from the sample pad. After the heat treatment is finished, the bridge can be brought into contact with the sample pad, for example via a blister button or similar means, allowing the heated liquid sample to flow to the assembly for detection. An exemplary heater can be an aluminum chip-based PTC thermostat heater, powered by a DC rechargeable battery. In an aspect, the heater can be selected to provide a heating temperature of 30 to 95° C., or 40 to 95° C.

In some aspects, it can be beneficial to pre-treat the optical probes prior to loading into the paper-based lateral flow assembly. Without wishing to be bound by theory, it is believed that such pre-treatment can improve stability of the probes and prevent or reduce non-specific absorption. In an aspect, suitable treatment can comprise contacting the optical probe with a treatment solution comprising basic buffer solution, stabilizing reagents, detergent, and a blocking agent. In a specific aspect, the treatment solution can comprise casein, a surfactant such as Tween 20, sucrose, trehalose, and trehalose in 50 mM borate buffer. A specific example of a treatment solution comprises 0.01 to 5 weight percent of aged casein, 0.01 to 2 weight percent Tween 20, 1 to 10 weight percent sucrose, 2 to 20 weight percent trehalose and balanced with 50 mM borate buffer, wherein weight percent is each component is based on the total weight of the treatment solution.

Another aspect of the present disclosure is a paper-based lateral flow strip assembly for multiplexed detection of multiple analytes in single strip. For example, FIG. 13 depicts multiplexed detection of three analytes in single strip. In this case, the detection unit comprises multiple (e.g., three) test lines functionalized with three different capture antibodies as shown in FIG. 13. Correspondingly, three types of optical probes are functionalized with three different detection antibodies. While the paper-based lateral flow strip assembly for multiplexed detection shown in FIG. 13 depicts three analytes, it should be noted that other quantities of test lines are also contemplated, for two or four test lines functionalized with two or four different capture antibodies, respectively.

Another aspect of the present disclosure is a paper-based lateral flow strip assembly for multiplexed detection of multiple analytes with a combination of individual strips, for example as shown in FIG. 14. As shown in FIG. 14, the combo-PLFS comprises two individual PLFS assemblies which share one sample pad for loading a single sample. Alternatively, the combination-PLFS can comprise three or four strips. Each strip can detect one to three analytes simultaneously. Accordingly, in an aspect, up to twelve analytes can be detected simultaneously using a combination PLFS assembly as described herein.

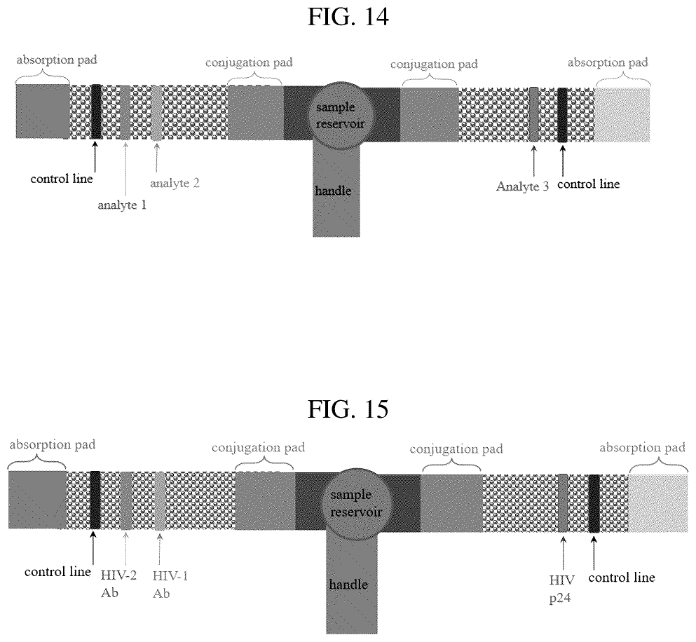

An example of a combo-PLFS for multiplexed detection of HIV p24 antigen, HIV-1 and HIV-2 antibodies is shown in FIG. 15. As shown in FIG. 15, the combination-PLFS comprises two individual PLFS assemblies which share one sample pad for loading a single sample. As shown in FIG. 15, one strip detects HIV p24 antigen and another strip detects HIV-1 and HIV-2 antibodies.

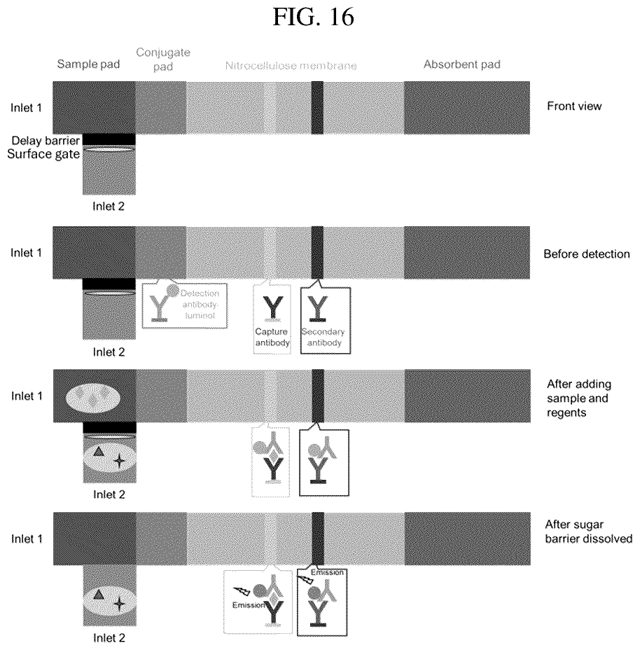

Another aspect of the present disclosure is a paper-based lateral flow strip assembly based on chemiluminescence, for example as shown in FIG. 16. When the PLFS assembly is to use chemiluminescence as the detection method, the optical probes are labelled with a detection moiety (also referred to as a “molecular recognition element”), preferably a detection antibody or aptamer which is further labeled with luminol (C₈H₇N₃O₂). As shown in FIG. 16, the PLFS is configured with a first and second inlet to provide sequential delivery of samples and reagents. The first inlet is used for sample loading, and the second inlet is used to provide chemiluminescence reagents such as ferricyanide and hydrogen peroxide. As shown in FIG. 16, the first and second inlets are connected by a delay barrier. Thus, during testing, samples will first flow to the conjugate pad and react with the detection antibody or aptamer, followed by capture by the capture antibody on the test line. Subsequently, the chemiluminescence reagents can flow into the main channel once the delay barrier is dissolved. Contact of the luminol with the chemiluminescence reagents on the test line will be activated by the chemiluminescence reagents, thereby emitting chemiluminescence, and the luminescence reagents intensity will depend on the analyte concentration. Variations for the components of the PLFS discussed above are also applicable to the present chemiluminescence embodiment.

The delay barrier can be, for example, a sugar barrier or a wax barrier. In an aspect, a sugar barrier can be provided by dispensing 0.5-5 μL of sugar (such as glucose, fructose and sucrose) solution to the desired location, and then drying to form the barrier. Alternatively, a wax barrier can be injected or printed to a desired location. As shown in FIG. 16, adjacent to the delay barrier a surface gate is provided. In an aspect, the surface gate can be provided by dropping 0.05-0.5 μL of 0.25-20 wt % surfactant solution (such as Tween-20, Triton-X 100 or Span-20) onto the desired location, and then drying.

Advantageously, the paper-based lateral flow strip assembly of the present disclosure can be used as a point-of-care testing tool for detection of biomarkers in human fluids (e.g., blood, plasma, scrum, saliva). For example, applications of the paper-based lateral flow strip assembly described herein include testing of various protein biomarkers such as traumatic brain injury (TBI) biomarkers (e.g., neuron Specific Enolase (NSE), ubiquitin C-terminal hydrolase-L1 (UCH-L1), glial fibrillary acidic protein (GFAP), S100 calcium-binding protein B (S100B)), HIV-1 biomarkers including p24 antigen, HIV-1 and HIV-2 antibodies, ovarian cancer biomarkers (cancer antigen 125 (CA125), carcinoembyonic antigen (CEA), human epididymis protein 4 (HE4)), sepsis biomarkers (C-reactive protein (CRP), procalcitonin (PCT), interleukin-6 (IL-6), haptoglobin and haptoglobin-related protein (Hp&HpRP)), Alzheimer's Disease biomarkers (amyloid precursor protein generates Amyloid-β (Aβ42, Aβ-40), neurofilament light chain product (NfL), GFAP), and SARS-CoV-2 viral load (nucleocapsid protein (N) and spike protein(S)). The paper-based lateral flow strip assembly can also be used for testing for various illicit drugs (e.g., cocaine, fentanyl, opioids, nuprenorphine, and the like). A significant improvement is therefore provided by the present disclosure.

This disclosure is further illustrated by the following examples, which are non-limiting.

### Examples

### Synthesis of Single Metal Core-Based Fluorescence Probes

To synthesize the nanostars, 0.5-1.5 wt % HAuCl₄·3H₂O aqueous was first diluted with water 10 times. 29-49 mM trisodium citrate was then added into the solution. Subsequently, freshly prepared 0.065-0.085 wt % NaBH₄ solution with 35-45 mM trisodium citrate was added into the above mixture and incubated under stirring at room temperature overnight to form a seed solution. Next, 40-60 mM HAuCl₄ aqueous solution was then mixed with 5-15 mM PVP in dimethylformamide (DMF); and the gold seed solution was mixed with 10 mM polyvinylpyrrolidone (PVP) at room temperature for 24 h. The latter was quickly added into the former one under stirring at room temperature, and then sitting for 13 h. To coat a silica space layer, tetraethyl orthosilicate (TEOS) and NH₄OH were added into the Au nanostar solution and then incubated for 12-24 h. The above particles were added into a (3-aminopropyl) trimethoxysilane (APTMS) ethanolic solution; and 10 mM CF405L with succinimidyl ester moity, such as Cyanine 7 and CF405L, were added to the nanoparticle. After washed with ethanol, tetraethyl orthosilicate (TEOS) and 28% ammonia hydroxide were added into the solution with 20 minutes of intervals. After incubation overnight, the silica-coated gold nanostars with dye encapsulated were formed. The resulting optical probes were washed and re-dispersed through centrifuging in ethanol solution.

### Synthesis of Single Metal Core-Based SERS Probes

The gold seed solution was prepared as described above. The gold seed solution was mixed with Raman reporter molecules, such as 2-4 mM 4-Mercaptobenzoic acid (4-MBA), to immobilize Raman reporters on the core surface. To coat a silica layer, tetraethyl orthosilicate (TEOS) and NH₄OH were added into the Au nanostar solution and then incubated for 12-24 h. The silica-coated gold nanostars (40-60 μg/mL) were suspended into ethanol. 80-120 μg/mL fluorescent dye, such as Cyanine 7 dyes, were added into the solution under stirring for 20 minutes. Tetraethyl orthosilicate (TEOS) and 28% ammonia hydroxide were added into the solution with 20 minutes of intervals. After incubation overnight, the silica-coated gold nanostars with dye encapsulated were formed.

### Synthesis of Bimetallic SERS Probes

An aqueous 20-30 mM HAuCl₄ was mixed with an aqueous 29-39 mM trisodium citrate solution. 0.070%-0.080% NaBH₄ was then injected into the solution, sitting overnight. Subsequently, poly(vinyl pyrrolidone) (PVP, MW10,000) was dissolved in the above seed solution with constant stir for another 24 h to stabilize gold seed solution. Next, the PVP-coated gold seed solution was injected into a 163-183 mM HAuCl₄ solution under continuous stirring. The colorless mixture gradually turned dark blue while stirring at room temperature overnight. The resulting Au nanostars were extracted in ethanol for bimetallic Au—Ag core synthesis. That is, the ethanol solution of Au nanostar was mixed with 0.05-0.15 M L-Ascorbic acid, 0.05-0.15 M AgNO₃ and 0.05-0.15 M NaOH while stirring at room temperature overnight to form bimetallic Au—Ag cores. To coat a silica layer, the bimetallic Au—Ag core solution was mixed with Raman reporter molecules, such as 2-4 mM 4-Mercaptobenzoic acid (4-MBA), to immobilize Raman reporters on the core surface. Subsequently tetraethyl orthosilicate (TEOS) and NH₄OH were added into the solution and then incubated for 12-24 h. The silica-coated metallic cores were separated and then mixed with 3-(triethoxysilyl) propylsuccinic anhydride (TEPSA) solution to form a silica outer-shell on the particle surface. To functionalize the silica surface with detection antibodies, the bimetallic core@Raman reporter@silica particles were mixed with a 5-15 mM phosphate-buffered saline (PBS) solution containing 5-15 mg/mL N-ethyl-N′-(3-(dimethylamino) propyl) carbodiimide/N-hydroxysuccinimide (EDC/NHS). Next, 0.5-2.0 mg/mL detection antibodies were added into the above liquid and incubated at 2-8° C. overnight. Subsequently, a 5-20% BSA solution added into the above solution, subject to centrifuging and washing with a 0.5-3% BSA PBS solution. For storage, the bimetallic optical probes were dispersed to a solution containing 0.5-3% 7-day aged casein, 0.10-0.3% Tween 20, 5-20% sucrose, 3-8% trehalose, and 40-60 mM borate.

### Synthesis of Bimetallic Fluorescence Probes

The bimetallic cores were prepared as described above. To coat a silica space layer, tetraethyl orthosilicate (TEOS) and NH₄OH were added into the Au nanostar solution and then incubated for 12-24 h. The silica-coated bimetallic cores (40-60 μg/mL) were suspended into ethanol. 80-120 g/mL fluorescent dye, such as Cyanine 7 dyes, were added into the solution under stirring for 20 minutes. Tetraethyl orthosilicate (TEOS) and 28% ammonia hydroxide were then added into the solution with 20 minutes of intervals. After incubation overnight, the bimetal core@silica@dye-encapsulated silica probes were formed. The resulting optical probes were washed and re-dispersed through centrifuging in ethanol solution.

### Preparation of PLFS

To fabricate an integrated paper lateral flow strip (PLFS), a large sized (e.g., comparable to an A4 paper size) nitrocellulose membrane laminated on an adhesive backing layer was selected. The capture antibodies and the secondary antibodies were dispensed onto the test line and the control line on the nitrocellulose membrane, respectively, by a dispensing machine with 3 mm of gap between the two lines. The dispensed nitrocellulose membrane was dried at room temperature for 1 h and stored at a dry state at 2-6° C. The membrane was then cut into individual strips (3 mm wide) by a cutter machine. Next, the plasma separation unit, conjugate pad, and absorbent pad were partially stacked on the adhesive backing layer. Each component was overlapped by 1.5-2 mm to ensure flow of liquid throughout the paper strip. After a PLFS with a dimension of 60 mm×3-4 mm was assembled, a solution containing the optical probe-detection antibody complex was dropped onto the conjugate pad, and then dried at room temperature for 1 h and stored at a dry state.

In an aspect of PLFS comprising a nano-array chip on the detection zone, the chip was be placed onto the adhesive backing layer and overlapped with the nitrocellulose membrane for 1.5-2 mm. The top of the chip was coated by a wetting cover containing a mixture of 5-30 mg of surfactants (such as Span-20, Tween-20) and 1-2 uL of sugar (such as glucose, fructose and sucrose). Afterwards, the PLFS was dried under an ambient condition for 0.5 hours.

### Results of Paper-Based Lateral Flow Strips

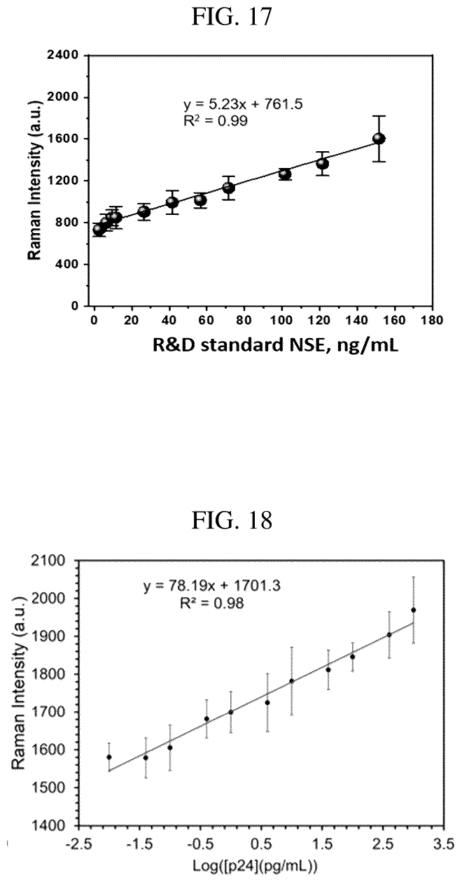

FIG. 17 shows the calibration curve of the PLFS device with the gold-based SERS probes for detection of NSE in 50% human serum diluted with PBS buffer. The limit of detection (LOD) of NSE in serum was 1.3 ng/mL. The fabrication details are described above. When testing a sample, a small volume (100 μL) of liquid was loaded onto the sample pad, waiting for 20 min. Subsequently, Raman spectra were collected from the test zone by a portable spectrometer under excitation by a 785 nm laser.

FIG. 18 shows the calibration curve of the PLFS with Au—Ag-based SERS probes for detection of p24 antigen in 10 mM PBS buffer and 50% plasma condition. This device showed a LOD as low as 88 fg/mL in 50% plasma with a linear range from 0.1 pg/mL to 1.0 ng/mL. The details of PLFS fabrication and testing are described as above.

The utility of the optical probes and lateral flow assemblies described herein have been further examined for testing of HIV antigen. To provide a system for detection of HIV antigens, gold nanostars were further modified with bulb-like silver particles to form plasmon hybridization and greatly increase SERS enhancement factor. Introduction of AuAgNST@4MBA@SiO₂-antip24 probes into the paper lateral flow strip (PLFS) increased the sensitivity of p24 detection compared to probes made of gold nanostars. Furthermore, a blood plasma separation filter was integrated into PLFS to decrease the interference of blood sample matrix, enabling the LOD of p24 detection in whole blood low to 0.05 pg/mL. The performance of PLFS with AuAgNST@4MBA@SiO₂-antip24 as the probes were further validated by preclinical seroconversion panel experiments. Given the blood plasma separation time was 10 s for 30 ul whole blood, the total detection can be completed within 30 mins.

Synthesis of Au nanostar and AuAg hybridized nanoparticles: 200 uL of 25 mM HAuCl₄ was diluted with 18 mL water and then 400 uL of 38.8 mM trisodium citrate was mixed with the solution, followed by injection of freshly prepared 200 uL of 0.075% NaBH₄. The reaction mixture was stirred constantly at room temperature overnight to prepare seed solution. Subsequently, 1.88 g PVP was dissolved in the above seed solution with constant stir for another 24 h to stabilize gold seed solution. To form gold nanostars, 23.8 UL of 173 mM HAuCl₄ solution was added into 15 mL of DMF containing 1.5 g PVP, followed by rapid injecting 43 μL of PVP-coated gold seed solution with stirring. The colorless mixture solution gradually turned into dark blue and continued to be stirred overnight at room temperature. The resulting Au nanostar solution was centrifuged and washed in ethanol for preparation of AuAg nanoparticles. 10 mL of Au nanostar ethanol solution was mixed with 70 uL of 0.1M L-Ascorbic acid, 15 uL of 0.1M AgNO₃ and 75 uL of 0.1M NaOH and stirred overnight at room temperature to form AuAg nanoparticles. The formed bimetal nanoparticles were collected and washed in ethanol for 3 times.

Functionalization of metal nanoparticles with anti-p24 antibodies: Modification of antibodies was carried out with carbodiimide chemistry. 1 mL of COOH-terminated Aunanostar@4MBA@SiO₂ and AuAg@4MBA@SiO₂ particles were activated by adding a 10 mM PBS solution with 10 μL 10 mg/mL EDC and NHS solutions. After incubating for 30 min, 20 uL 1.0 mg/mL anti-p24 antibody (SAB3500882) was slowly added into the mixture solution and stood overnight in 40C. After that, 100 ul 10% BSA solution was added to block the non-specific site on the surface, followed by centrifuging and washing using PBS containing 1% BSA solution. Finally, the antibody-functionalized metal nanoparticles were suspended in 2 mL probe solution (1% 7-day aged casein, 0.25% Tween 20, 10% sucrose, 5% trehalose, 50 mM borate) and stored in 4° C. for later use.

To coat silica layers, 10 mL of gold nanostar solution and AuAg hybridized solution were mixed with 120 uL of 3 mM 4MBA, respectively, for 30 min to ensure the full cover of Raman reporters on the particle surface. Then, 60 uL of TEOS and 60 uL of NH₄OH were added into the solution successively. After 24 hours, the silica-coated metal particles were centrifuged and resuspended in ethanol, followed by incubating with 40 uL of TEPSA solution to label —COOH group on the silica surface. The above solution was washed 3 times and resuspended in 4 mL water for future use.

Fabrication of paper lateral flow strip (PLFS): PLFS included a sample pad (17×3 mm), a conjugate pad (7×3 mm), nitrocellulose membrane (NC membrane, 25×3 mm) and an absorbent pad. The sample pad was treated with Tris-HCl buffer (0.05 M, pH 8.0) containing 0.23% of Triton X-100 and 0.15 M NaCl. Then it was dried at 37° C. for 1 h and stored in desiccator at room temperature. The glass fiber was employed as the conjugate pad which was immersed in probe solutions with antibody-functionalized metal nanoparticles. After that, conjugate pad was dried at 37° C. for 1 h and stored in 4° C. 25 uL capture antibody (MAB8790) and secondary antibody at concentration of 0.3 mg/mL and 1 mg/mL, respectively, were stripped onto the NC membrane by a dispenser. The formed T line was 8 mm from upstream the edge of the NC membrane, while C line was 13 mm from that of the NC membrane. The membrane was then dried at 37° C. for 1 h. The prepared pads were placed on the adhesive backing card with 2 mm overlap for each two components to ensure the solution migrate through the whole strip. An automatic paper cutter (Autokun) was used to cut strips to a 3 mm width. These prepared paper strips were used for detecting antigens in buffer and 50% plasma condition. When testing, 100 uL running buffer (10 mM PBS with 0.07% Tween-20) with a certain concentration of analyte was dropped onto the sample pad. As for the blood test, a blood filtration membrane (LF-D23-X1, I.W. Tremont Co., Inc.) with the dimension of 16×3 mm was placed at the most upstream, followed by a sample pad (7×3 mm) saturated with Tris-HCl buffer. The remaining structure was same as the above.

Seroconversion panel test: The used panels were AccuVert™ HIV-1 Seroconversion Panel 0600-0271, 0600-025 and 0600-0272 purchased from LGC Clinical Diagnostics, UK. Each panel consists of serial plasma collected from an individual during the development of an HIV infection. These samples were tested on our SERS-PLFS and FDA-licensed assays including Bio-Rad GS HIV Combo Ag/Ab Enzyme immunoassay (EIA) and Abbott Determine™ HIV-1/2 Ag/Ab Combo. The whole experiments were conducted according to the manufacturers' instructions in IALS Clinical Testing Center, University of Massachusetts Amherst. ELISA cutoff was calculated by adding Calibrator absorbance value to 0.200 according to the manufacturers' instructions. PLFS cutoff was set as the SERS signal of blank samples (limit of blank) according to the calibration curve derived in plasma condition.

Finite Difference Time Domain Simulations. Commercial FDTD software (Lumerical) was used to study the extinction spectra, surface polarization charge distribution, and electric fields. SERS probe. A total-field scattered-field (TFSF) was used as the input light source. A mesh size of 1 nm was imposed in the simulation region. The perfectly matched layer (PML) boundary conditions were considered in the simulation. Electric fields were calculated as E/E₀, where E is the local electric field intensity and E₀ is the excitation electric field intensity. The excitation wavelength was set as 785 nm.

Star-shaped metal nanoparticles exhibited high SERS signals due to the concentrated electromagnetic field at the sharps. It was reported that Au nanostar (AuNST) showed almost three orders of SERS enhancement factors higher than Au nanospheres, leading to a LOD of 0.86 ng/mL for detection of neuron-specific enolase (NSE) biomarker when using Au nanostar-derived SERS probes. To further increase the sensitivity of SERS probes for the detection P24 antigen, silver-gold hybridized nanostructure was adapted by post-modification of Au nanostar particles since Ag has higher plasmon than Au identical structure. The Au nanostar had a core of 35 nm in diameter with spikes of 20 nm in length. Silver formed a bulb-like structure with a diameter of around 25 nm on the surface of gold nanostar. The distribution of Ag and Au was further confirmed by EDX mapping. Then a silica layer was formed on the outer surface of nanoparticles to encapsule Raman reporters, prevent metal nanostars from aggregating as well as provide additional zone for antibody modifications. The thickness of such silica layer on the surface of AuNST and AuAgNST was 3-5 nm.

Blood tests were performed by replacing the cellulose pad with a blood plasma filter membrane. Since plasma constitutes around 55% of total blood, the maximum plasma volume can be achieved after separation is around 16.5 uL when total blood sample is 30 uL, and some of them may be retained in the filter membrane. Glass fiber was then used as the sample pad next to the filter membrane in order to reduce retaining of samples owing to their low bed volume. It was also treated with buffer solutions to enable even distribution of sample to the following conjugation pad as well as stabilize the buffer condition of samples. After assembling blood plasma separation filter, sample pad and conjugation pad coated with SERS probes, NC membrane and absorption pad, the integrated PLFS can be directly used for testing HIV p24 antigen spiked in whole blood.

The results indicated that such PLFS integrated with AuAg nanostar-derived SERS probes can be used for P24 detection in both whole blood and plasma. They have potential to serve as POC for patients' self-tests at home, as well as monitoring P24 level in clinics.

In summary, a SERS based PLFS has been successfully developed for detection of HIV p24 antigen. The SERS probes were fabricated using AuAg bimetal nanostar as plasmon core with SERS enhancement factor 5.4 higher compared to pure Au nanostar which was determined by experiment and FDTD simulations. The generated SERS-PLFS can achieve a LOD low to 1.4 fg/mL and 0.09 pg/mL in buffer and 50% plasma condition, respectively. With a blood plasma separation unit, the strips were successfully applied to whole blood testing and allowed a LOD of 0.05 pg/mL using only one drop of blood (30 uL) with a linear range from 0.1 pg/mL to 1 ng/mL. Such high sensitivity can effectively discriminate and detect HIV antigen at an early stage. The sensitivity and feasibility of SERS-PLFS was further validated by seroconversion panel. It displayed an ultrasensitive response to HIV p24 antigen, even comparable to 4^{th} generation ELISA. The sensor engineered in this disclosure has the great potential to improve sensitivity of current rapid HIV P24 antigen tests and enable early diagnosis of HIV at home or source-limited areas.

This disclosure further encompasses the following aspects, which are non-limiting.

Aspect 1: An optical probe comprising: a plasmonic core comprising a nanoparticle, the nanoparticle comprising gold, silver, copper, or a combination thereof; a buffer layer on a surface of the plasmonic core, wherein the buffer layer is transparent and insulating; and a composite shell on the buffer layer, the composite shell comprising a plurality of fluorophores embedded in a transparent metal oxide matrix.

Aspect 2: The optical probe of aspect 1, wherein the plurality of fluorophores comprises organic dyes, inorganic semiconducting quantum dots, chalcogenides, graphene oxide quantum dots, carbon dots, metal-organic framework (MOF), or a combination thereof.

Aspect 3: An optical probe comprising: a plasmonic core comprising a nanoparticle, the nanoparticle comprising gold, silver, copper, or a composition thereof; a transparent shell on the plasmonic core, the transparent shell comprising a transparent ternary metal oxide; and a plurality of Raman dyes proximate to the plasmonic core and embedded in the transparent shell.

Aspect 4: The optical probe of aspect 3, wherein the transparent ternary metal oxide is derived from a compound of the formula MₓSi₁₋ₓO₂, or M₂ₓAl₂₋ₓO₃ wherein x is 0 to 0.5 and M is Mg, Ba, Sr, Ca, K, Na, or a combination thereof.

Aspect 5: The optical probe of aspect 3 or 4, wherein plurality of Raman dyes comprises malachite green (MG), mercaptobenzoic acid (MBA), Rhodamine 6G, IR1061, or a combination of thereof.

Aspect 6: The optical probe of any of aspects 1 to 5, wherein the nanoparticle of the plasmonic core comprises nanospheres, nanocubes, nanorods, nanostars, or a combination thereof, optionally wherein the nanoparticle is porous.

Aspect 7: The optical probe of any of aspects 1 to 6, wherein the plasmonic core comprises gold.

Aspect 8: The optical probe of any of aspects 3 to 7, wherein the plasmonic core comprises gold and silver.

Aspect 9: The optical probe of aspect 8, wherein the plasmonic core comprises gold nanostars comprising silver nanodots on the surface of the nanostars, or wherein the bimetallic plasmonic core comprises bimetallic gold/silver nanocubes.

Aspect 10: An optical probe comprising: a plasmonic core comprising a nanoparticle, the nanoparticle comprising gold or silver; and a plurality of secondary nanoparticles proximate to the plasmonic core to provide plasmonic coupling by charge transfer, provided that when the plasmonic core comprises gold, the secondary nanoparticles comprise silver; and when the plasmonic core comprises silver, the secondary nanoparticles comprise gold.

Aspect 11: The optical probe of aspect 10, wherein the nanoparticle of the plasmonic core comprises nanospheres, nanocubes, nanorods, nanostars, or a combination thereof, optionally wherein the nanoparticle is porous.

Aspect 12: The optical probe of any of aspects 1 to 11, wherein the plasmonic core has a diameter of 10 to 200 nanometers, preferably 15 to 180 nanometers, preferably 15 to 70 nanometers.

Aspect 13: The optical probe of any of aspects 1 to 2 or 6 to 9, wherein the buffer layer has a thickness of 1 to 40 nanometers, preferably 2 to 20 nanometers.

Aspect 14: The optical probe of any of aspects 1 to 2, 6 to 9, and 12 to 13, wherein the composite shell has a thickness of 1 to 25 nanometers, preferably 2 to 20 nanometers.

Aspect 15: The optical probe of any of aspects 3 to 9 or 12, wherein the transparent shell has a thickness of 1 to 25 nanometers, preferably 2 to 20 nanometers.

Aspect 16: An article comprising the optical probe of any of aspect 1 to 15.

Aspect 17: A method of detecting a target species, the method comprising: introducing an analyte mixture to a detection system comprising the optical probe of any of aspects 1 to 15 or the article of aspect 16.

Aspect 18: A plasmonic nanoarray-containing a biosensor, the plasmonic nanoarray-containing a biosensor comprising: the optical probe of any of aspects 1 to 15, wherein the optical probe is functionalized with a plurality of molecular recognition elements; a plasmonic nano-array pattern or a plasmon gap mode pattern, each functionalized with a plurality of capture antibodies; wherein the optical probe is capable of binding to the plasmonic nano-array pattern or the plasmon gap mode pattern upon contact with an analyte.

Aspect 19: The plasmonic nanoarray-containing biosensor of aspect 18, wherein the plurality of molecular recognition elements comprises antibodies, aptamers, or small molecules, preferably antibodies.

Aspect 20: The plasmonic nanoarray-containing biosensor of aspect 18 or 19, wherein the plasmonic nano-array pattern comprises a nano-disc array pattern, nano-sphere array pattern, nano-triangle pattern, nano-rod pattern, nano-ring pattern, nano-hole pattern, or nano-cylinder pattern on a solid substrate; the nanoarray comprises gold, silver, copper or a combination thereof; and the nanoarray is deposited on a solid substrate comprising silicon, glass, quartz, silica, or a metal oxide.

Aspect 21: The plasmonic nanoarray-containing biosensor of aspect 18 or 19, wherein the plasmon gap mode pattern comprises a nano-array pattern on a fluorescence layer, a metal layer on the fluorescence layer, and a substrate on the metal layer; the nano-array pattern comprises a nano-disc array pattern, nano-sphere array pattern, nano-triangle pattern, nano-rod pattern, nano-ring pattern, nano-hole pattern, or nano-cylinder pattern; the nano-array comprises gold, silver, copper, or a combination thereof; the fluorescence layer comprises metal-organic frameworks (MOFs), 2D chalcogenides, silica, alumina, or a metal oxide, for example a fluorophore-decorated metal oxide; the metal layer comprises gold, silver, copper or a combination thereof; and the substrate comprises silicon, glass, quartz, silica, or a metal oxide.

Aspect 22: A method of detecting a target species, the method comprising: introducing an analyte mixture comprising the target species to a detection system comprising the plasmonic nanoarray-containing biosensor of any of aspects 18 to 21.

Aspect 23: A paper-based lateral flow strip assembly comprising the optical probe of any of aspects 1 to 15 or the plasmonic nanoarray-containing biosensor of any of aspects 18 to 21.

Aspect 24: The paper-based lateral flow strip assembly of aspect 23 comprising: a plasma separation unit; a conjugation pad comprising a plurality of optical probes, each according to of any of aspects 1 to 15, wherein the optical probes are labelled with a detection moiety, preferably a detection antibody; a membrane, preferably a microfluidic membrane, for example a nitrocellulose membrane; a detection unit comprising a capture antibody capable of interacting with an analyte, wherein the analyte is capable of binding to the optical probe, and a control line comprising a secondary antibody; and an absorbent pad adjacent to the membrane.

Aspect 25: The paper-based lateral flow strip assembly of aspect 24, wherein the detection unit comprises the plasmonic nanoarray-containing biosensor of any of aspects 18 to 21, and further comprising a wetting cover, wherein the wetting cover comprises surfactants, sugar, or a combination thereof.

Aspect 26: The paper-based lateral flow strip assembly of aspect 24 or 25, wherein the plasma separation unit comprises a filtration membrane, preferably wherein the filtration membrane is treated with an aqueous salt solution, preferably wherein the aqueous salt solution has a concentration of 0.05 to 2 M, and wherein the salt comprises KCl, NaCl, CaCl₂), KNO₃, NaNO₃, Ca(NO₃)₂, KSO₄, NaSO₄, K₃PO₄, Na₃PO₄, K₂HPO₄, Na₂HPO₄, or a combination thereof.

Aspect 27: The paper-based lateral flow strip assembly of aspect 24 or 25, wherein the plasma separation unit comprises a porous electrospun nanofiber membrane treated with oxygen plasma, preferably wherein the porous electrospun nanofiber membrane comprises polymer nanofibers, more preferably wherein the polymer nanofibers comprise polyvinylpyrrolidone, poly(ethylene glycol), poly(ethylene oxide), poly(lactide), polylactic-co-glycolic acid, poly(vinyl alcohol), poly(ε-caprolactone), polysulfone, or a combination thereof.

Aspect 28: The paper-based lateral flow strip assembly of any of aspects 24 to 27, further comprising a heater positioned prior to the plasma separation unit.

Aspect 29: The paper-based lateral flow strip assembly of any of aspects 24 to 28, comprising two or more, preferably 2 to 4, test lines in the detection unit for detection of multiple analytes.

Aspect 30: The paper-based lateral flow strip assembly of any of aspects 24 to 29, wherein the paper-based lateral flow strip assembly is a combination assembly comprising more than one paper-based lateral flow strip sharing a single sample pad.

Aspect 31: The paper-based lateral flow strip assembly of any of aspects 24 to 30, wherein the detection moiety comprises a detection antibody labelled with luminol, and wherein the paper-based lateral flow strip assembly further comprises a second inlet to provide a chemiluminescence reagent to the assembly, wherein the second inlet is separated from a first inlet by a delay barrier comprising a sugar or a wax.

Aspect 32: A method of detecting an analyte, the method comprising: contacting a human fluid sample, preferably a whole blood sample, a blood plasma sample, a saliva sample, a nasal swab sample, or a urine sample, with the paper-based lateral flow strip assembly of any of aspects 24 to 31.

Aspect 33: The method of aspect 32, wherein the analyte is an HIV-1 biomarker comprising HIV p24 antigen or HIV-1 and HIV-2 antibodies in the blood plasma sample or the whole blood samples.

Aspect 34: The method of aspect 32, wherein the analyte is a traumatic brain injury biomarker comprising neuron specific enolase, ubiquitin C-terminal hydrolase-L1, glial fibrillary acidic protein, or S100 calcium-binding protein B in the blood plasma sample or the whole blood sample.

Aspect 35: The method of aspect 32, wherein the analyte is an ovarian cancer biomarker comprising cancer antigen 125, carcinoembryonic antigen, or human epididymis protein 4 in the blood plasma sample or the whole blood sample.

Aspect 36: The method of aspect 32, wherein the analyte is a sepsis biomarker comprising C-reactive protein, procalcitonin, interleukin-6, or haptoglobin and haptoglobin-related protein in the blood plasma sample or the whole blood sample.

Aspect 37: The method of aspect 32, wherein the analyte is an Alzheimer's Disease biomarker comprising amyloid precursor protein, Amyloid-β peptides, neurofilament light chain product, or glial fibrillary acidic protein in the blood plasma sample or the whole blood sample.

Aspect 38: The method of aspect 32, wherein the analyte is an illicit drug such as fentanyl, cocaine, opioids, or buprenorphine.

Aspect 39: The method of aspect 32, wherein the analyte is a SARS-COV-2 viral protein comprising nucleocapsid protein or spike protein in the saliva sample or the nasal swab samples.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through carbon of the carbonyl group.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. An optical probe comprising:
a plasmonic core comprising a nanoparticle, the nanoparticle comprising gold, silver, copper, or a combination thereof;
a buffer layer on a surface of the plasmonic core, wherein the buffer layer is transparent and insulating; and
a composite shell on the buffer layer, the composite shell comprising a plurality of fluorophores embedded in a transparent metal oxide matrix, wherein the plurality of fluorophores comprises organic dyes, inorganic semiconducting quantum dots, chalcogenides, graphene oxide quantum dots, carbon dots, metal-organic framework (MOF), or a combination thereof.

2. The optical probe of claim 1, wherein the nanoparticle of the plasmonic core comprises gold nanospheres, gold nanocubes, gold nanorods, gold nanostars, or a combination thereof, optionally wherein the nanoparticle is porous;
wherein the buffer layer has a thickness of 1 to 40 nanometers; and
wherein the composite shell has a thickness of 1 to 25 nanometers.

3. An optical probe comprising:
a plasmonic core comprising a nanoparticle, the nanoparticle comprising gold, silver, copper, or a composition thereof;
a transparent shell on the plasmonic core, the transparent shell comprising a transparent ternary metal oxide derived from a compound of the formula MₓSi₁₋ₓO₂, or M₂ₓAl₂₋ₓO₃ wherein x is 0 to 0.5 and M is Mg, Ba, Sr, Ca, K, Na, or a combination thereof; and
a plurality of Raman dyes proximate to the plasmonic core and embedded in the transparent shell.

4. The optical probe of claim 3,
wherein the nanoparticle of the plasmonic core comprises nanospheres, nanocubes, nanorods, nanostars, or a combination thereof, optionally wherein the nanoparticle is porous;
wherein the plasmonic core comprises gold or a combination of gold and silver, and having a LSPR band in a near-infrared I window of 700-900 nm or a near-infrared II window of 1000-1300 nm;
wherein plurality of Raman dyes comprises malachite green (MG), mercaptobenzoic acid (MBA), Rhodamine 6G, IR1061, or a combination of thereof; and
wherein the transparent shell has a thickness of 1 to 25 nanometers.

5. The optical probe of claim 4,
wherein the plasmonic core comprises gold nanostars comprising silver nanodots on the surface of the nanostars, or
wherein the bimetallic plasmonic core comprises bimetallic gold/silver nanocubes.

6. An optical probe comprising:
a plasmonic core comprising a nanoparticle, the nanoparticle comprising gold or silver; and
a plurality of secondary nanoparticles proximate to the plasmonic core to provide plasmonic coupling by charge transfer, provided that
when the plasmonic core comprises gold, the secondary nanoparticles comprise silver; and
when the plasmonic core comprises silver, the secondary nanoparticles comprise gold.

7. The optical probe of claim 6, wherein the nanoparticle of the plasmonic core comprises nanospheres, nanocubes, nanorods, nanostars, or a combination thereof, optionally wherein the nanoparticle is porous, wherein the plasmonic core has a diameter of 10 to 200 nanometers.

8. An article comprising the optical probe of claim 1.

9. A method of detecting a target species, the method comprising: introducing an analyte mixture to a detection system comprising the optical probe of claim 1.

10. A plasmonic nanoarray-containing a biosensor, the plasmonic nanoarray-containing a biosensor comprising:
the optical probe of claim 1, wherein the optical probe is functionalized with a plurality of molecular recognition elements comprising antibodies, aptamers, or small molecules;
a plasmonic nano-array pattern or a plasmon gap mode pattern, each functionalized with a plurality of capture antibodies;
wherein the optical probe is capable of binding to the plasmonic nano-array pattern or the plasmon gap mode pattern upon contact with an analyte.

11. The plasmonic nanoarray-containing biosensor of claim 10, wherein
the plasmonic nano-array pattern comprises a nano-disc array pattern, nano-sphere array pattern, nano-triangle pattern, nano-rod pattern, nano-ring pattern, nano-hole pattern, or nano-cylinder pattern on a solid substrate;
the nanoarray comprises gold, silver, copper or a combination thereof; and
the nanoarray is deposited on a solid substrate comprising silicon, glass, quartz, silica, or a metal oxide.

12. The plasmonic nanoarray-containing biosensor of claim 11, wherein
the plasmon gap mode pattern comprises a nano-array pattern on a fluorescence layer, a metal layer on the fluorescence layer, and a substrate on the metal layer;
the nano-array pattern comprises a nano-disc array pattern, nano-sphere array pattern, nano-triangle pattern, nano-rod pattern, nano-ring pattern, nano-hole pattern, or nano-cylinder pattern;
the nano-array comprises gold, silver, copper, or a combination thereof;
the fluorescence layer comprises metal-organic frameworks (MOFs), 2D chalcogenides, silica, alumina, or a metal oxide, for example a fluorophore-decorated metal oxide;
the metal layer comprises gold, silver, copper or a combination thereof; and
the substrate comprises silicon, glass, quartz, silica, or a metal oxide.

13. A method of detecting a target species, the method comprising: introducing an analyte mixture comprising the target species to a detection system comprising the plasmonic nanoarray-containing biosensor of claim 11.

14. A paper-based lateral flow strip assembly comprising an optical probe, comprising:
a plasma separation unit;
a conjugation pad comprising a plurality of optical probes, each according to claim 1, wherein the optical probes are labelled with a detection moiety, preferably a detection antibody;
a membrane;
a detection unit comprising a capture antibody capable of interacting with an analyte, wherein the analyte is capable of binding to the optical probe, and
a control line comprising a secondary antibody; and
an absorbent pad adjacent to the membrane;
optionally comprising two or more test lines in the detection unit for detection of multiple analytes.

15. The paper-based lateral flow strip assembly of claim 14, wherein the detection unit comprises a plasmonic nanoarray-containing biosensor comprising:
the optical probe, wherein the optical probe is functionalized with a plurality of molecular recognition elements;
a plasmonic nano-array pattern or a plasmon gap mode pattern, each functionalized with a plurality of capture antibodies;
wherein the optical probe is capable of binding to the plasmonic nano-array pattern or the plasmon gap mode pattern upon contact with an analyte, and
further comprising a wetting cover, wherein the wetting cover comprises surfactants, sugar, or a combination thereof.

16. The paper-based lateral flow strip assembly of claim 14, wherein the plasma separation unit comprises a filtration membrane, or wherein the plasma separation unit comprises a porous electrospun nanofiber membrane treated with oxygen plasma.

17. The paper-based lateral flow strip assembly of claim 14, wherein the detection moiety comprises a detection antibody labelled with luminol, and wherein the paper-based lateral flow strip assembly further comprises a second inlet adjacent to the sample pad to provide a chemiluminescence reagent to the assembly, wherein the second inlet is separated from the sample pad by a delay barrier comprising a sugar or a wax.

18. The paper-based lateral flow strip assembly of claim 14, wherein the optical probe is contacted with a treatment solution prior to loading into the paper-based lateral flow strip assembly, wherein the treatment solution comprises a basic buffer solution, a stabilizing reagent, a detergent, and a blocking agent.

19. A method of detecting an analyte, the method comprising:
contacting a human fluid sample, preferably a whole blood sample, a blood plasma sample, a saliva sample, a nasal swab sample, or a urine sample, with the paper-based lateral flow strip assembly of claim 14.

20. The method of claim 19,
wherein the analyte is an HIV-1 biomarker comprising HIV p24 antigen or HIV-1 and HIV-2 antibodies in the blood plasma sample or the whole blood samples; or
wherein the analyte is a traumatic brain injury biomarker comprising neuron specific enolase, ubiquitin C-terminal hydrolase-L1, glial fibrillary acidic protein, or S100 calcium-binding protein B in the blood plasma sample or the whole blood sample; or
wherein the analyte is an ovarian cancer biomarker comprising cancer antigen 125, carcinoembryonic antigen, or human epididymis protein 4 in the blood plasma sample or the whole blood sample; or
wherein the analyte is a sepsis biomarker comprising procalcitonin (PCT), C-reactive protein, soluble triggering receptor expressed on myeloid cells-1 (sTREM-1), interleukin-6, or haptoglobin and haptoglobin-related protein in the blood plasma sample or the whole blood sample; or
wherein the analyte is an Alzheimer's Disease biomarker comprising amyloid precursor protein, Amyloid-β peptides, neurofilament light chain product, or glial fibrillary acidic protein in the blood plasma sample or the whole blood sample; or
wherein the analyte is an illicit drug such as fentanyl, cocaine, opioids, or buprenorphine; or
wherein the analyte is a SARS-CoV-2 viral protein comprising nucleocapsid protein or spike protein in the saliva sample or the nasal swab samples; or
wherein the analyte is a sepsis disease biomarkers comprising procalcitonin (PCT), C-reactive protein (CRP), interleukin-1 (IL-1), soluble triggering receptor expressed on myeloid cells (sTREM)-1 in the blood plasma sample or the whole blood sample; or
wherein the analyte is Hepatitis C biomarkers comprising Hepatitis C virus (HCV) core antigen, anti-HCV in the blood plasma sample or the whole blood sample.
